(19) United States
(12) Patent Application Publication
(10) Pub. No.: US 2010/0030117 A1
(43) Pub. Date: Feb. 4, 2010

(54) SYSTEMS AND METHODS FOR MONITORING ERECTILE FUNCTION AND DIAGNOSING ERECTILE DYSFUNCTION

(75) Inventors: Luya Li, Coquitlam (CA); Sidney Ben Radomski, Toronto (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: Urodynamix Technologies Ltd., Vancouver (CA)

(21) Appl. No.: 12/525,078

(22) PCT Filed: Feb. 2, 2008
(86) PCT No.: PCT/CA08/00204

§ 371 Date: Aug. 26, 2009

Related U.S. Application Data
(60) Provisional application No. 60/888,012, filed on Feb. 2, 2007.

(51) Int. Cl. A61B 5/103

(52) U.S. Cl. 600/587

(57) ABSTRACT

Methods and systems for monitoring erectile function or dysfunction provide for determining concentration data of biochemical compounds in a subject's erectile tissues; and analyzing the concentration data to yield values indicative of the subject's erectile function. The concentration of biochemical compounds may be measured using Near Infrared Spectroscopy (NIRS). The biochemical compounds may comprise at least one compound from the group consisting of Hemoglobin, Oxygenated Hemoglobin, Cytochromes and Myoglobin.

### CROSS-REFERENCE TO RELATED APPLICATION

This application claims priority from United States patent application No. 60/888012 filed Feb. 2, 2007 and entitled SYSTEMS AND METHODS FOR DIAGNOSING ERECTILE DYSFUNCTION. For purposes of the United States of America, this application claims the benefit under 35 U.S.C. § 119 of United States patent application No. 60/888012 filed Feb. 2, 2007 and entitled SYSTEMS AND METHODS FOR DIAGNOSING ERECTILE DYSFUNCTION which is hereby incorporated herein by reference.

### TECHNICAL FIELD

This invention relates to methods and systems for monitoring erectile function and diagnosing erectile dysfunction.

### BACKGROUND

Erectile dysfunction (“ED”) affects a significant section of the male population. Some available methods for diagnosing ED involve:
using Doppler ultrasound to measure blood flow in the penis; or
using snap gauges or strain gauges to measure changes in penile circumference.
   A disadvantage of ultrasonic Doppler measurements is that it can be hard to maintain intimate contact between an ultrasound probe and the penis to obtain measurements over an extended period. It is also difficult to keep an ultrasound probe positioned so as to obtain consistent measurements over an extended period. Because of these issues, it is difficult to use ultrasonic monitoring to monitor a subject overnight.

Near Infrared Spectroscopy (“NIRS”) is a technique which involves emitting near infrared (“NIR”) light and receiving the NIR light after it has passed through a tissue or other medium of interest. NIRS can be applied to study and monitor biochemical compounds in the body. Emitted NIR light penetrates skin and other tissues and some of it is absorbed by biochemical compounds which have an absorption spectrum in the NIR region. NIR light which is not absorbed is scattered. Each biochemical compound has a different absorption spectrum. It is possible to estimate the concentration of biochemical compounds in the tissues by measuring characteristics of NIR light that has been detected after it has passed through the tissues.

There is a need for practical and cost-effective systems for measuring erectile function and diagnosing erectile dysfunction.

### SUMMARY

This invention provides methods and systems for monitoring erectile function.

One aspect of the invention provides methods which monitor erectile function by measuring the concentration of biochemical compounds in tissues of the penis or clitoris through the use of NIRS. The biochemical compounds may comprise one or more compounds from the group consisting of deoxygenated hemoglobin (Hb), Oxygenated Hemoglobin (HbO₂), Cytochromes (Cyt), and Myoglobin (Mb). Trends in the concentration data can be correlate to changes in erectile state. An analysis of trends in the concentration data may be used to diagnose erectile dysfunction.

Another aspect of the invention provides systems for monitoring erectile function and/or diagnosing erectile dysfunction. The systems comprise a data monitoring subsystem which processes and analyses concentration data of biochemical compounds in tissues of the penis or clitoris or adjacent tissues. The concentration data may be obtained through a data acquisition subsystem, such as a NIRS subsystem. The biochemical compounds which are monitored may include at least one compound from the group consisting of Hb, HbO₂, Cyt and Mb. The data monitoring subsystem stores concentration data at periodic intervals. The data monitoring subsystem monitors trends in the concentration data and may perform other analyses of the concentration data.

Further aspects of the invention and features of specific embodiments of the invention are described below.

### BRIEF DESCRIPTION OF THE DRAWINGS

In drawings which illustrate non-limiting embodiments of the invention,

FIG. 1 is a flow chart illustrating a method for monitoring erectile function;

FIG. 2 is a plot showing a concentration of oxygenated hemoglobin in a subject's penis as a function of time;

FIG. 2A is an example plot which includes a curve which indicates the concentration of HbO₂ during the onset of an erection in a subject having normal erectile function;

FIG. 3 is a block diagram illustrating a system for monitoring erectile function;

FIG. 3A is a schematic illustration of a display in an example embodiment of the invention;

FIG. 4 is a block diagram illustrating a specific implementation of the system of FIG. 3;

FIG. 5 is a block diagram illustrating a data monitoring subsystem which may be used in the system of FIG. 4; and,

FIG. 6 is a block diagram of a system for monitoring erectile function that includes a separable data acquisition and collection system.

### DESCRIPTION

Throughout the following description, specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

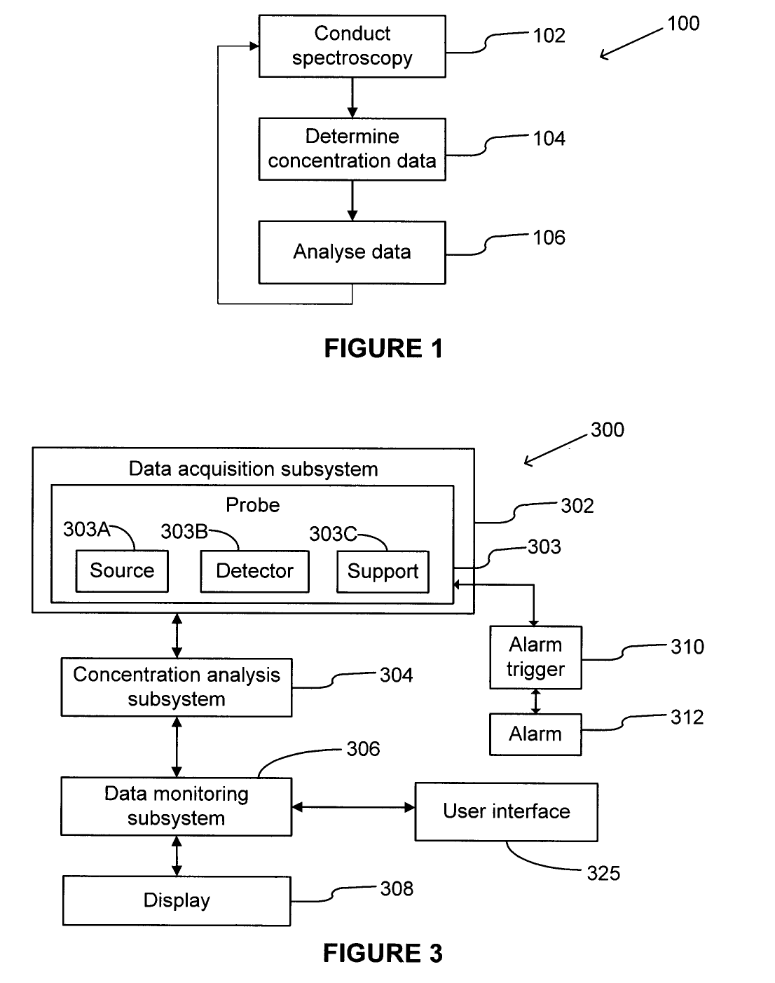

FIG. 1 illustrates a method 100 for monitoring erectile function. At block 102, spectroscopy, preferably absorption spectroscopy, is conducted on a subject to detect and measure concentrations of one or more biochemical compounds in the tissues of the subject's penis or clitoris. NIRS is a form of absorption spectroscopy which may be used for detecting biochemical compounds which have an absorption spectrum in the NIR region.

In one embodiment of the invention, NIRS may be conducted on a subject by directing NIR light at the skin of the subject's penis or clitoris, and detecting and measuring the NIR light that is scattered back through the skin. As discussed in further detail below, this may be achieved by placing an NIR transmitter and an NIR receiver close together on the surface of the skin, so as to detect NIR back scattered light from the tissues of the subject's penis or clitoris.

Method 100 proceeds to block 104, where the scattered light that is detected at block 102 is analysed to obtain concentration data for biochemical compounds in the tissues. The compounds may comprise one or more compounds from the group consisting of Hb, HbO₂, Cyt, and Mb, for example.

The concentration data is analysed at block 106. The analysis may comprise monitoring trends in the data, for example, monitoring a change in concentration value relative to an initial concentration value, or monitoring the first derivative of the concentration with respect to time. Such trends (particularly trends in the concentration of Hb or HbO₂ or a function of one or more of the concentrations of Hb and HbO₂) generally correlate to changes in erectile state. Therefore, based on an analysis of these trends, one can obtain information about the subject's erectile performance.

The steps described above may be repeated continuously for so long as it is desired to monitor the subject. In some embodiments, monitoring may be performed over an extended period, such as overnight.

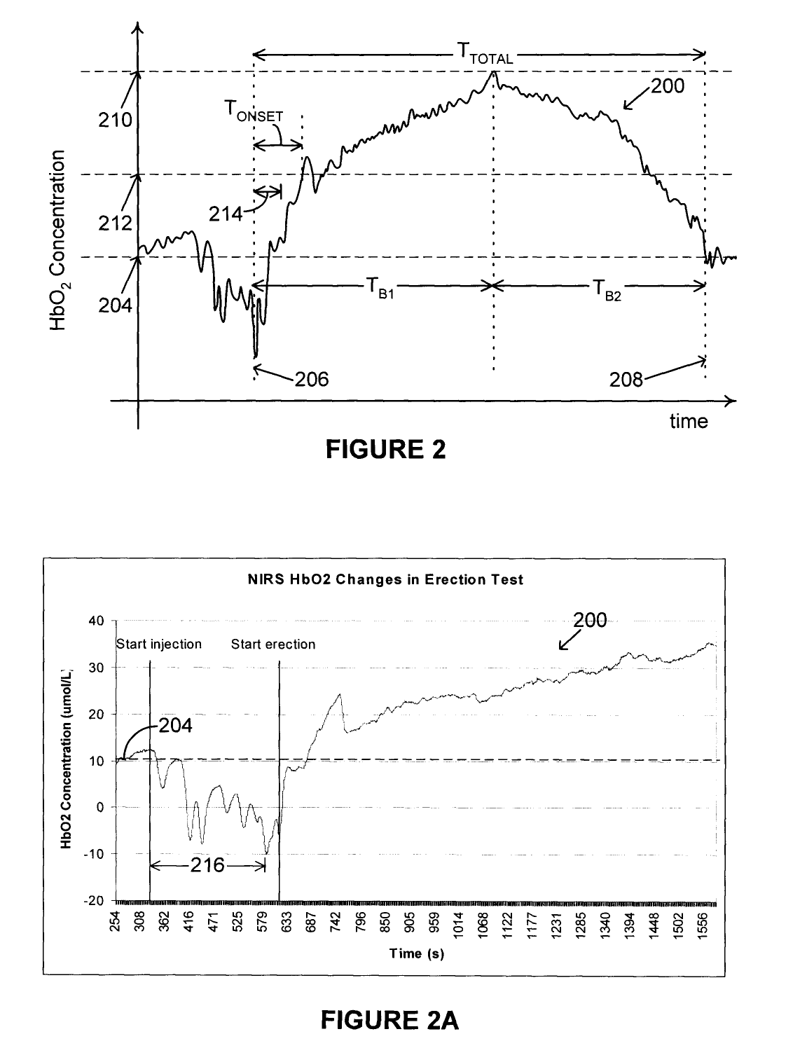

FIG. 2 is a plot which includes a curve 200 which indicates the concentration of HbO₂ in tissues of a subject's penis as a function of time. Curve 200 is typical of a subject having normal erectile function and experiencing an erection. Curve 200 is a schematic illustration and does not represent actual data. A number of features of curve 200 may be measured. Such measurements may be performed automatically by a system according to the invention as described in more detail below.

Examples of features of curve 200 that may be measured include:
a baseline level 204 of the concentration of HbO₂ in the absence of an erection;
a total erection time T_{Total} which may be measured between an onset time 206 at which the concentration first drops below the baseline level 204 by more than a threshold amount, and an end time 208 at which the concentration returns to the baseline level after having risen to a peak 210 of curve 200 that is above the baseline level;
a peak concentration, which may be measured as the difference between a value of the concentration at peak 210 during the erection and baseline value 204;
a “half-time” TB₁ between onset time 206 and the time of peak 210;
a “half-time” TB₂ between the time of peak 210 and end time 208;
an onset time Tonset between onset time 206 and a time at which the concentration exceeds a threshold value 212;
an onset rate, which may be measured as the first derivative of the concentration during a period 214 just after onset time 206;
an area under curve 200 during all of or a selected part of the erection.

FIG. 2A is a plot which includes a curve 200 which indicates the concentration of HbO₂ during the onset of an erection in a subject having normal erectile function. As shown in FIG. 2A, at the onset of the erection, the concentration measured by curve 200 dips below baseline 204 during a period 216 and then increases to values greater than baseline 204. The method may detect the onset of an erection by monitoring the concentration over time and identifying this signature (a dip in concentration followed by an increase in concentration).

Onset time 206 and end time 208 may be used to represent an approximation of the times of the onset and end of an erection, respectively. However, a different determination may be used to approximate the onset and end of an erection. For example, the time of onset may be determined to be the time at which the concentration of HbO₂ has the overall lowest value of curve 200.

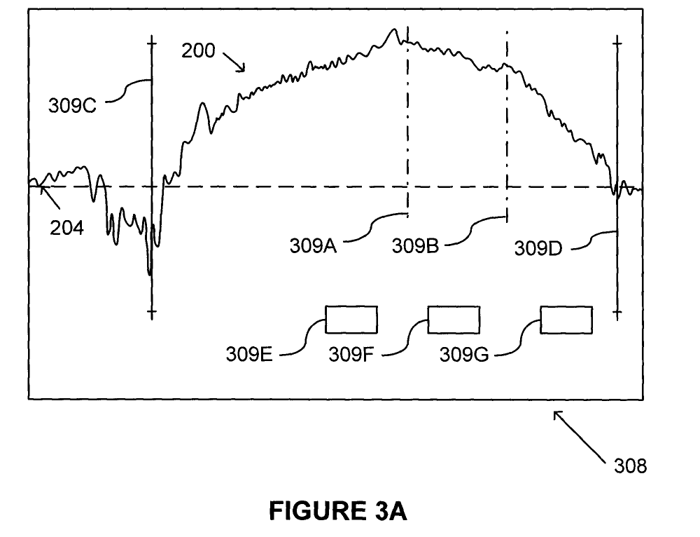

FIG. 3 illustrates a system 300 for monitoring erectile function. System 300 comprises a data acquisition subsystem 302 to detect biochemical compounds in the tissues. Data acquisition subsystem 302 may use absorption spectroscopy techniques, for example, NIRS, to detect biochemical compounds. Subsystem 302 may comprise one or more sources 303A of NIR light and one or more detectors 303B for the NIR light. subsystem 302 may also comprise a support 303C for holding the source(s) 303A and detector(s) 303B in place on the subject's anatomy. Source(s) 303A, detector(s) 303B and support 303C (if present) may be described as a probe 303.

Any suitable means may be used to hold light source(s) 303A and light detector(s) 303B against the subject's anatomy. For example:
In some embodiments, support 303C comprises a wrap that can be wrapped around the subject's penis.
In some embodiments, support 303C may comprise an adhesive patch which can hold source(s) 303A and/or detector(s) 303B against the subject's penis. The light source(s) and detector(s) may be located such that light from the light source(s) is transmitted through the subject's penis and received at the light detector(s); the light is back scattered in tissues of the subject's penis and received at the light detector(s) or some combination thereof. The light source(s) and detector(s) may be located at any suitable location along the subject's penis. In some embodiments, sets of light sensor(s) and light detector(s) are placed at multiple locations along the subject's penis.

Data acquisition system 302 may comprise a commercially-available NIRS system connected to receive data from probe 303, for example.

The data which is acquired by data acquisition subsystem 302 is analysed by a concentration analysis subsystem 304 to determine the concentration of one or more biochemical compounds in the tissues or some function thereof. The compounds may comprise at least one compound from the group consisting of Hb, HbO₂, Cyt, and Mb. It is not necessary that the concentration of any of these compounds be determined in any particular units.

The concentration data is input to a data monitoring subsystem 306 which stores the data at periodic intervals. Data monitoring subsystem 306 analyses the data to detect parameters that may be indicative of erectile function or dysfunction (the parameters may include one or more of the features of curve 200 that are discussed above).

Data monitoring subsystem 306 is connected to a display 308 for displaying the data. Data monitoring subsystem 306 may also be connected to a user interface 325 which a user can operate to input instructions to data monitoring subsystem 306 and to control the output to display 308. For example, a user may input instructions to:
calculate and display one or more of the parameters that may be indicative of erectile function or dysfunction;
zoom in to or zoom out from a portion of curve 200 visible on display 308;
scroll forward or backward in time along curve 200;
determine and display the time between selected features of curve 200, for example, the time between the onset and end of an erection; or
provide an assessment of whether the data indicates normal or abnormal erectile function.
   Moveable cursors on display 308 may be provided to facilitate such user input.

In an example embodiment, shown in FIG. 3A, display 308 displays curve 200 and includes one or more cursors that can be moved by operating user interface 325 to measure times between different portions of curve 200 and/or differences in concentration between different points on curve 200 and/or between a point on curve 200 and baseline level 204. In the illustrated embodiment, two cursors 309A and 309B are shown. Display 308 also has marks 309C and 309D respectively indicating the start and end points of the erection as determined by data monitoring subsystem 306. In the illustrated embodiment, display 308 displays values for a number of parameters 309E, 309F, 309G that are calculated by data monitoring subsystem 306.

Data monitoring subsystem 306 may also be connected to a user interface 325 which allows a user to perform such functions as:
manipulating one or more cursors;
highlighting portions of a concentration curve;
zooming in on a selected portion of a concentration curve;
entering notes or other information about the subject;
storing data;
retrieving comparison data and optionally displaying the comparison data on display 308 to permit comparison of curve 200 to the comparison data; and
the like.
   The results may be output to display 308.

System 300 may incorporate an alarm 312 that may comprise, for example, an audible alarm (e.g. bell or beep), visual alarm (e.g. light), a tactile alarm (e.g. a vibrator) or a combined alarm. Alarm 312 may be actuated automatically by an alarm trigger 310 if system 300 fails to detect a reliable signal from probe 303 (as could be the case if probe 303 somehow came off of the subject's penis or other erectile tissue). Alarm trigger 310 may be wired to alarm 312 or it may transmit a wireless message which activates alarm 312 on a wireless receiving device (e.g. Personal Digital Assistant, pager, or cellular phone). Other types of alarms are possible.

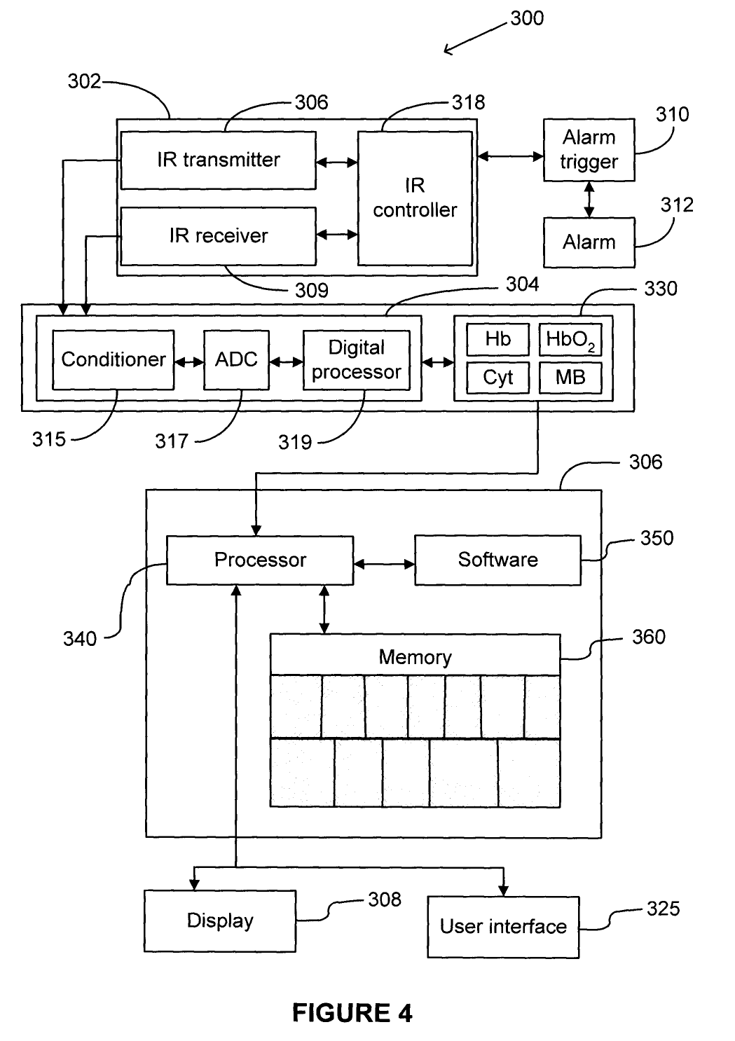

FIG. 4 illustrates a specific implementation of system 300. Data acquisition subsystem 302 is provided to conduct NIRS on a patient. Data acquisition subsystem 302 comprises an NIR transmitter 306 and an NIR receiver 309, each connected to an NIR controller 318. Although only one NIR receiver is illustrated, data acquisition subsystem 302 may comprise more than one NIR receiver 309. In some embodiments, NIR transmitter 306 comprises an optical fibre that carries NIR light to the subject by way of an optical fiber or other light guide. This can be beneficial if the light source operates at a temperature such that it would be noticeable or uncomfortable to a subject.

Preferably, NIR transmitter 306 and NIR receiver 309 are contained in a probe or probes placed on the patient's skin. NIR transmitter 306 directs NIR light at the patient's skin. The NIR light may have one or more bands in the spectrum range of 700 to 950 nm. The transmitted NIR light penetrates the skin and other tissues and some of it is absorbed by biochemical compounds, such as proteins, which each have a different absorption spectrum in the NIR region. The NIR light which is not absorbed is either scattered back through the skin or transmitted through the tissues to skin on the far side of the subject's penis or clitoris. Some of this back scattered and/or transmitted light is detected by NIR receiver 309.

NIR controller 318 may determine the intensity of the NIR light transmitted by NIR transmitter 306.

The NIR light which is detected and received by NIR receiver 309 is output by NIR receiver 309 in the form of an analog signal. This signal is sent to concentration analysis subsystem 304. A signal conditioner 315 conditions the analog signal to prepare it for analog to digital conversion by converter 317. For example, signal conditioner 315 may amplify and/or filter the signal at the frequencies of interest.

After the conditioned analog signal is converted to a digital signal by converter 317, digital processor 319 may perform further filtering of the signal, such as to remove signals attributable to background NIR radiation.

Digital processor 319 analyses the signal to determine concentration data 330 for biochemical compounds. The compounds may comprise at least one compound from the group consisting of Hb, HbO₂, Cyt, and Mb. Each of these biochemical compounds absorbs NIR light at a different spectrum. Thus, by comparing the spectrum of the NIR light transmitted by NIR transmitter 306 with the spectrum of the NIR light received by NIR receiver 309, concentration data 330 may be determined. For example, concentration data 330 may be determined by transmitting NIR light having a set of discrete wavelengths, and monitoring the wavelengths contained in the output signal of NIR receiver 309. Concentration data 330 is then sent to data monitoring subsystem 306.

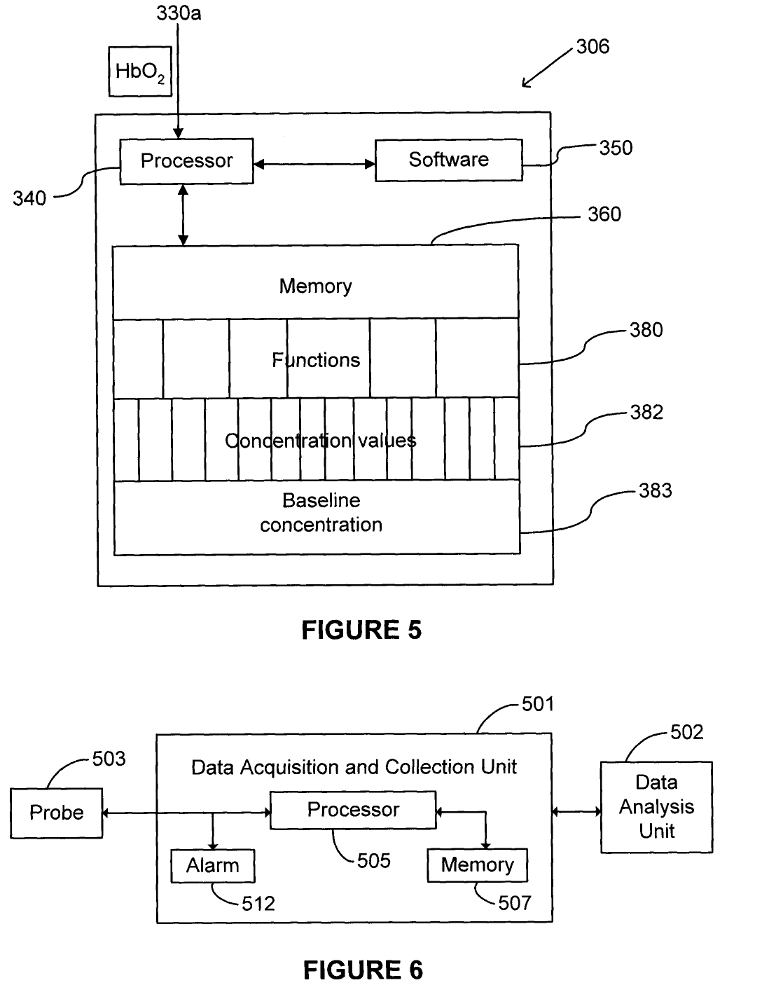

FIG. 5 illustrates, in further detail, data monitoring subsystem 306. Data monitoring subsystem 306 receives a subset of concentration data 330, namely concentration data 330A for HbO₂. Although not illustrated, data monitoring subsystem 306 may also receive concentration data 330 for one or more of Hb, Cyt and Mb. In some embodiments, parameters indicative of erectile function or dysfunction may involve concentrations or concentration trends of two or more of HbO₂, Hb, Cyt and Mb.

Data monitoring subsystem 306 comprises a processor 340, which executes instructions contained in software 350 and reads/writes data to/from memory 360. Memory 360 stores, for example, a plurality of functions 380 which can compute parameters relating to an erection from concentration data 330A obtained during the erection. Memory 360 may also store a baseline concentration value 383 of concentration data 330A, and a number of values 382 of concentration data 330A, recorded at periodic intervals, such as a few times each second, once every few seconds or once every few minutes. In some embodiments, measurements are made at a rate of at least 0.5 Hz. In some embodiments the rate of measurements is in the range of 0.1 Hz to 100 Hz.

Processor 340 calls and executes functions in software 350 with selected information from memory 360 as inputs to the functions.

Although not illustrated, a device for printing out information may be provided. The device may print information displayed by display 308 or other information related to the subject's erectile status.

Software 350 may include functions for automatically detecting and counting erections; functions for automatically computing parameters indicative of a subject's erectile function; and/or functions for collecting statistical information regarding a subject's erections.

In some embodiments, as illustrated, for example, in FIG. 6, apparatus according to the invention comprises a data acquisition and collection unit 501 that is separable from a data analysis unit 502. Data acquisition and collection unit 501 may comprise a relatively small battery-powered unit that connects to a probe 503 to acquire NIRS data during a monitoring period (such as overnight). Data acquisition and collection unit 501 may comprise an alarm 512 that vibrates, makes a sound, or otherwise alerts a subject if a signal from probe 503 is not detected. A processor 505 manages the operation of data acquisition and collection unit 501 and stores NIRS data in a memory 507.

After collection unit 501 has collected data during one or more monitoring periods, the data can be uploaded to data analysis unit 502. Data analysis unit 502 may comprise a specialized unit or a personal computer or the like executing suitable software. Data analysis unit 502 can analyze the data that has been collected by data acquisition and collection unit 501 as described above. In some embodiments, data analysis unit 502 can download to data acquisition and collection unit 501 operating parameters that affect the operation of data acquisition and collection unit 501. The parameters may include, for example, a rate of data acquisition.

Certain implementations of the invention comprise data processors which execute software instructions which cause the data processors to perform a method of the invention. For example, one or more processors in an apparatus for monitoring erectile function may implement the methods of FIG. 1 by executing software instructions in a program memory accessible to the data processors. The data processors may comprise one or more microprocessors, embedded processors, computer systems, digital signal processors or the like. The invention may also be provided in the form of a program product. The program product may comprise any medium which carries a set of computer-readable instructions which, when executed by one or more data processors, cause the one or more data processors to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like. The computer-readable instructions on the program product may optionally be compressed or encrypted.

As will be apparent to those skilled in the art in the light of the foregoing disclosure, many alterations and modifications are possible in the practice of this invention without departing from the spirit or scope thereof. For example, an apparatus may be provided which contains one or more subsystems or devices described above, such as a data monitoring subsystem 306, display 308, alarm trigger 310 and alarm 312. Some embodiments described herein may provide more than one data processor. In other embodiments, the functions of two or more such data processors are combined in one data processor.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method for monitoring erectile function in a mammal, the method comprising:
non-invasively monitoring a concentration of one or more biochemical compounds in erectile tissues of the mammal; and,
detecting an erection of the erectile tissues based on changes in the monitored concentration.

2. A method according to claim 1 wherein the one or more biochemical compounds are selected from the group consisting of hemoglobin, oxygenated hemoglobin, cytochromes and myoglobin.

3. A method according to claim 1 wherein non-invasively monitoring comprises transmitting infrared light into the erectile tissues, receiving infrared light that has passed through the erectile tissues, and determining the concentration of the one or more biochemical compounds by performing near-infrared spectrometry using the received infrared light.

4. A method according to claim 3 wherein the infrared light comprises light in the wavelength range of 700 nm to 950 nm.

5. A method according to claim 3 wherein the erectile tissues comprise erectile tissues of a penis of the mammal, transmitting infrared light comprises holding an infrared light source against skin of the penis and receiving infrared light comprises holding an infrared light detector against the skin of the penis.

6. A method according to claim 5 wherein the received infrared light comprises light that has been back-scattered within the penis.

7. A method according to claim 5 wherein the received infrared light comprises light that has been transmitted through the penis.

8. A method according to claim 1 further comprising computing a first derivative of the concentration as a function of time.

9. A method according to claim 1 wherein the biochemical compound comprises oxygenated hemoglobin and the method comprises detecting an onset of an erection by identifying a pattern comprising a decrease in the concentration of the oxygenated hemoglobin from a baseline level followed by an increase in the concentration of the oxygenated hemoglobin to above the baseline level.

10. A method according to claim 1 wherein the biochemical compound comprises oxygenated hemoglobin and the method comprises establishing a baseline level for the concentration of the oxygenated hemoglobin in the absence of an erection.

11. A method according to claim 10 comprising detecting an onset of an erection based on changes in the concentration of oxygenated hemoglobin and detecting a peak concentration of the oxygenated hemoglobin during the erection.

12. A method according to claim 11 comprising measuring a first half-time between the onset of the erection and a time corresponding to the peak concentration of oxygenated hemoglobin.

13. A method according to claim 11 comprising detecting an end time of the erection based on changes in the concentration of oxygenated hemoglobin subsequent to the onset and measuring a second half-time between a time corresponding to the peak concentration of oxygenated hemoglobin and the end time of the erection.

14. A method according to claim 10 comprising detecting an onset of an erection and an end time of the erection based on changes in the concentration of oxygenated hemoglobin and integrating the oxygenated hemoglobin concentration during at least a part of the erection.

15. A method according to claim 10 comprising detecting an onset of an erection, detecting a threshold-crossing time after the onset at which the concentration of oxygenated hemoglobin crosses a threshold and determining a time difference between the onset and the threshold-crossing time.

16. A method according to claim 10 comprising detecting an onset of an erection, detecting an end time of the erection and determining a time difference between the onset and the end time.

17. A method according to claim 11 wherein detecting the onset comprises detecting a minimum in the concentration of oxygenated hemoglobin.

18. A method according to claim 5 wherein holding the infrared light source against skin of the penis comprises wrapping a wrap around the penis.

19. A method according to claim 5 wherein holding the infrared light source against skin of the penis comprises adhesively affixing a patch to the penis wherein the light source is affixed to the patch.

20. A method according to claim 1 comprising storing a record of the concentration as a function of time and subsequently displaying a plot of the concentration as a function of time on a display.

21. A method according to claim 20 comprising automatically determining an onset time for an erection and displaying on the display indicia identifying the onset time.

22. A method according to claim 20 comprising automatically determining an end time for an erection and displaying on the display indicia identifying the end time.

23. A method according to claim 3 comprising, automatically triggering an alarm upon making a determination that a signal representing the received infrared light is unreliable.

24. A method according to claim 1 comprising monitoring the concentration periodically at a rate of M Hz or higher.

25. A method according to claim 1 comprising continuing non-invasively monitoring for a period of time and automatically detecting based upon the monitored concentration, and counting, a plurality of erections occurring during the period of time.

26. A method according to claim 2 comprising monitoring a concentration of at least two biochemical compounds selected from the group consisting of hemoglobin, oxygenated hemoglobin, cytochromes and myoglobin.

27. A method according to claim 1 wherein non-invasively monitoring is performed while the mammal is asleep.

28. A method according to claim 3 wherein non-invasively monitoring comprises logging data derived from the received infrared light that has passed through the erectile tissues in a collection unit worn on the mammal and subsequently transferring the logged data to a data analysis unit.

29. A method according to claim 28 comprising, at the collection unit, generating an alarm if a signal corresponding to the received infrared light is not detected.

30. A method according to claim 1 comprising outputting derived information relating to one or more erections of the mammal by at least one of printing the derived information, displaying the derived information or storing the derived information in a memory.

31. A system for monitoring erectile function in a mammal, the system comprising:
a data acquisition system comprising a near-infrared light source, and a near-infrared light receiver generating a signal indicative of near-infrared light received at the receiver;
a concentration analysis subsystem configured to analyze the signal to compute concentration data for one or more biochemical compounds;
and a data monitoring subsystem configured to identify concentration data indicative of an erection and to determine one or more parameters of the erection from the concentration data.

32. A system according to claim 31 wherein the concentration analysis subsystem is configured to compute concentration data for oxygenated hemoglobin.

33. A system according to claim 32 wherein the data monitoring subsystem is configured to detect an onset of an erection by identifying a pattern comprising a decrease in the concentration of the oxygenated hemoglobin from a baseline level followed by an increase in the concentration of the oxygenated hemoglobin to above the baseline level.

34. A system according to claim 33 wherein the data monitoring subsystem is configured to record a time of a minimum in the concentration of oxygenated hemoglobin as an erection onset time.

35. A system according to claim 31 wherein the concentration analysis subsystem is configured to compute concentration data for two or more biochemical compounds selected from the group consisting of hemoglobin, oxygenated hemoglobin, cytochromes and myoglobin.

36. A system according to claim 31 concentration analysis subsystem is configured to compute concentration data at least one biochemical compound selected from the group consisting of hemoglobin, oxygenated hemoglobin, cytochromes and myoglobin.

37. A system according to claim 31 wherein the data acquisition system and concentration analysis system compose a near-infrared spectrometer.

38. A system according to claim 31 wherein the biochemical compound comprises oxygenated hemoglobin and the data monitoring subsystem is configured to establish a baseline level for the concentration of the oxygenated hemoglobin in the absence of an erection.

39. A system according to claim 38 wherein the data monitoring subsystem is configured to detect an onset of an erection based on changes in the concentration of oxygenated hemoglobin and to detect a peak concentration of the oxygenated hemoglobin during the erection.

40. A system according to claim 39 wherein the data monitoring subsystem is configured to measure a first half-time between the onset of the erection and a time corresponding to the peak concentration of oxygenated hemoglobin.

41. A system according to claim 39 wherein the data monitoring subsystem is configured to detect an end time of the erection based on changes in the concentration of oxygenated hemoglobin subsequent to the onset and to measure a second half-time between a time corresponding to the peak concentration of oxygenated hemoglobin and the end time of the erection.

42. A system according to claim 38 wherein the data monitoring subsystem is configured to detect an onset of an erection and an end time of the erection based on changes in the concentration of oxygenated hemoglobin and integrate the oxygenated hemoglobin concentration during at least a part of the erection.

43. A system according to claim 38 wherein the data monitoring subsystem is configured to detect an onset of an erection, detect a threshold-crossing time after the onset at which the concentration of oxygenated hemoglobin crosses a threshold and determine a time difference between the onset and the threshold-crossing time.

44. A system according to claim 38 wherein the data monitoring subsystem is configured to detect an onset of an erection, detect an end time of the erection and determine a time difference between the onset and the end time.

45. A system according to claim 31 comprising a memory wherein the concentration analysis system is configured to periodically log the concentration data in the memory.

46. A system according to claim 45 wherein the concentration analysis system is configured to log the concentration data in the memory a plurality of times each second.

47. A system according to claim 31 wherein the concentration analysis system and data monitoring subsystem comprise software processes executing on a data processor.

48. A system according to claim 47 wherein the data monitoring subsystem comprises a function for counting erections.

49. A system according to claim 48 wherein the data monitoring subsystem comprises a function for computing statistical information regarding the counted erections.

50. A system according to claim 31 wherein the near-infrared light source comprises an optical fiber to carry light from a light emitter to a location against the skin of the mammal.

51. A system according to claim 31 comprising a wrap to hold the near-infrared light source and near-infrared light receiver against a penis of the mammal.

52. A system according to claim 31 comprising an adhesive patch to hold the near-infrared light source and near-infrared light receiver against a penis of the mammal.

53. A system according to claim 31 comprising a display wherein the system is configured to display a curve representing the concentration data as a function of time.

54. A system according to claim 53 wherein the system is configured to display on the display the one or more parameters of the erection.

55. A system according to claim 31 comprising at least one additional near-infrared light source, and at least one additional near-infrared light receiver.

56. A system according to claim 31 comprising means for outputting derived information relating to one or more erections of the mammal by at least one of printing the derived information, displaying the derived information or storing the derived information in a memory.

57. A system according to claim 56 wherein the derived information comprises a time difference between an onset of the erection and a feature of the concentration data.

58. A system according to claim 57 wherein the feature of the concentration data comprises a time of a maximum concentration.

59. A system according to claim 57 wherein the feature of the concentration data comprises an end time of the erection.

60. A system according to claim 57 comprising means for integrating the concentration data for the time period between the onset of the erection and the feature of the concentration data.

61. (canceled)

62. (canceled)
